(19) United States
(12) Patent Application Publication
(10) Pub. No.: US 2015/0272967 A1
(43) Pub. Date: Oct. 1, 2015

(54) EXTREMELY HIGH LOADING DOSE

(71) Applicant: Frank J. Grasso, Cleveland, OH (US)

(72) Inventor: Frank J. Grasso, Cleveland, OH (US)

(21) Appl. No.: 14/584,036

(22) Filed: Dec. 29, 2014

Related U.S. Application Data
(60) Provisional application No. 61/922,290, filed on Dec. 31, 2013.

(51) Int. Cl. A61K 31/593; A61K 31/592

(52) U.S. Cl. CPC A61K 31/593; A61K 31/592

(57) ABSTRACT

Methods including novel dosing methods and/or dosage forms and compositions of Vitamin D compounds and/or mimics thereof alone and/or in combination with other agents for the treatment of proliferative conditions and/or cancers in humans.

### CROSS-REFERENCE TO RELATED APPLICATIONS

This applicant claims the benefit of the United States of America provisional patent application Ser. No. 61/922,290 filed 2013 Dec. 31 by the present inventor.

### STATEMENT REGARDING FEDERALLY SPONSORED RESEARCH

Not applicable.

### BACKGROUND OF THE INVENTION

Various attempts have been made to use Vitamin D compounds for prevention and treatment of cancer and sometimes limited beneficial effects have been achieved. In a clinical trial with prostate cancer patients who were administered Vitamin D3 in an amount significantly lower than the Vitamin D formulation amount provided by the present invention (55%) showed improvement, by a significant decrease in the number of positive cancer biopsy samples. Marshall, D et al J Clin Endocrinol Metab. Jul 2012; 97(7): 2315-2324.In a clinical trial with breast cancer patients with supplementation of modest low doses of Vitamin D compared to the dose amount provided by the present invention the patients who received the Vitamin D supplementation had a significantly improved disease free survival than did those without the supplementation (32.6 months vs. 25.5) Median overall survival was (43.8 months in the Vitamin D group vs. 32.8 months in the group without supplementation Zeichner SB et al Clin Breast Cancer. 2014 Aug 15. pii: S1526-8209(14)00166-9. 1 patient with a grade III brain tumor and 10 patients astrocytoma grade IV who were treated with a Vitamin D analogue at a low dose of Vitamin D compared to the dose amount provided by the present invention. The results included an objective response that 3 of all the patients showed a remarkable radiological progressive and durable regression of the lesions approaching near total disappearance and their complete clinical remission had been observed from 4, 5 and 7 years respectfully after the initiation of the Vitamin D therapy. The median overall survival time for the 10 glioblastoma patients was 21 months. Trouillas P et al. J Neurooncol. 2001 January;51(1):57-66. After diagnosis for astrocytoma grade IV, the median life expectancy ranges from under a year or up to about 14 months.

The present invention is concerned with methods including novel dosing methods and/or dosage forms and compositions of Vitamin D compounds and/or mimics thereof alone and/or in combination with other agents for the treatment of proliferative conditions and/or cancers in humans.

Vitamin D is a generic term for a family of secosteroids that have affinity for the Vitamin D-Binding Protein and/or Vitamin D Receptor, and is involved in the physiologic regulation of calcium and phosphate metabolism.

Vitamin D3 is naturally synthesized in human skin from 7-dehydrocholesterol and ultraviolet light and Vitamin D3, or the analog Vitamin D2, can be ingested from the food or supplements and either are contained in a number of multivitamin and/or calcium supplements.

Vitamin D compounds exist in several forms and 25-hydroxyergocalciferol and calcidiol are the two specific Vitamin D compounds that are measured in serum to determine a person's Vitamin D status.

Deficiencies of Vitamin D can cause rickets and osteomalacia. Vitamin D3 and D2 have been used to prevent rickets and osteomalcia and to treat the same and calcidiol has been used to treat metabolic bone disease associated with chronic renal failure.

The Institute of Medicine of the National Academy of Sciences has concluded the Tolerable Upper Intake Level (UL) for Vitamin D is 100 mcg per day, based on evidence that higher doses are associated with an increased risk of hypercalciuria, hypercalcemia and related sequelae, including cardiac arrhythmias, seizures, and generalized vascular and other soft-tissue calcification. Standing Committee on the Scientific Evaluation of Dietary Reference Intakes, Dietary reference intakes: calcium, phosphorus, magnesium, Vitamin D, and fluoride. Washington, DC: National Academy Press (2011).

Hypercalcemia is a frequent finding in cancer patients and can be observed in any type of cancer. 1007 Soyfoo M S, Support Care Cancer. 2013 May;21(5):1415-9. doi: 10./s00520-012-1683-5. Epub 2012 December. Several cancers, including those originating in the breast, prostate, and lung, exhibit a propensity to metastasize to bone, resulting in debilitating skeletal complications and the sequelae includes hypercalcemia which is one of the most serious metabolic disorders associated with cancer. Lipton A., Semin Oncol. 2010 October;37 Suppl 2:S15-29.10.1053/j.seminonco1.2010.10.002;Muggia F M, Semin Oncol 1990 April;17(2 Suppl 5):3-9. Hypercalcemia is not only one of the most serious and common metabolic disorders associated with cancer and also is the most common life-threatening metabolic disorder associated with neoplastic diseases and preventive measures that is taught for patients with cancer by the National Cancer Institute at the National Institute of Health provides for cautious use or elimination of drugs that may complicate management including medications that contain Medical research concerning Vitamin D has turned to its therapeutic effects in a variety of pathological conditions including a number of proliferative diseases and cancers with addressing the issue and/or problem of the innate nature of Vitamin D compounds which can cause hypercalcemia and/or unmanageable and/or unsustainable hypercalcemia.

Vitamin D compounds while potentially useful in retarding tumor growth have the disadvantage that they are very potent calcemic agents that cause elevated blood calcium levels by stimulating intestinal calcium absorption and bone calcium resorption. Accordingly, there has been a desire in the art in regard to treatment of proliferative diseases and/or cancers in humans with Vitamin D compounds for methods and/or compositions including dosing methods and compositions without concomitant enhancement of unsustainable and/or unmanageable calcemic activity.

There have been efforts to explore the activity of vitamin D compounds in patients with certain cancers including daily escalation dosing. In prostate cancer, this approach was tested and only minimal dose escalation above physiologic replacement doses was possible before hypercalcemia and/or hypercalcuria developed. Dose escalation was limited by hypercalcemia in the vast majority of patients. Osborn J L, Urol Oncol, 1995; 1:195-8 An alternative to escalating dosing was attempted to avoid hypercalcemia by pulse dosing, and periodic non-daily dosing at 45 ug of a Vitamin D compound was tried with patients having metastatic castration-resistant prostate cancer, however hypercalcemia was significantly higher and more deaths were noted in the Vitamin D compound arm, and the trial was halted. Scher H I, J Clin Oncol 2011,29:2191-2198.

There has a long felt need for more effective treatments for cancers and proliferative conditions. There also has been a long felt need for effective treatments for cancers and proliferative conditions for which there are no effective treatments.

### BRIEF SUMMARY OF THE INVENTION

The invention is a method of treatment comprising the administration a Vitamin D compound and/or mimics thereof in a daily dose amount of 0.25 mg to 500 gm and preferably from 1.25 mg to 500 mg and even more preferably from 2.5 mg to 350 mg and most preferably from 12.5 mg to 50 mg to humans with proliferative conditions and/or cancers.

Another embodiment of the invention is a method of treatment comprising the administration a Vitamin D compound and/or mimics thereof in a daily dose amount of 0.25 mg to 500 gm and preferably from 1.25 mg to 500 mg and even more preferably from 2.5 mg to 350 mg and most preferably from 12.5 mg to 50 mg to humans with proliferative conditions and/or cancers and if hypercalcemia and/or unmanageable and/or unsustainable hypercalcemia develops before the treatment is satisfactorily successful and/or adequate and once hypercalcemia is resolved, then low daily doses ranging from lmcg or less to 0.25 mg or from 1 mcg to 1.25 mg or more of Vitamin D compound and/or mimics thereof is administered to said patient for treatment and then said low daily dose can be gradually escalated to the loading dose amounts as recited herein which is then administered for treatment to said patient until the treatment is successful and/or adequate or until hypercalcemia and/or unmanageable and/or unsustainable hypercalcemia redevelops and this method can be repeated as necessary.

The invention further includes compositions comprising a Vitamin D compounds and/or mimics thereof in daily dose amount of 0.25 mg to 500 gm and preferably from 1.25 mg to 500 mg and even more preferably from 2.5 mg to 350 mg and most preferably from 12.5 mg to 50 mg, alone, and/or in combination with other agents for treatment of humans with proliferative conditions and/or cancers.

The invention is also a dosage form comprising a Vitamin D compound and/or mimics thereof administered to humans with proliferative conditions and/or cancer for treatment in a daily dose amount of 0.25 mg to 500 gm and preferably from 1.25 mg to 500 mg and even more preferably from 2.5 mg to 350 mg and most preferably from 12.5 mg to 50 mg.

The preferred Vitamin D compounds useful in the present invention include calcidiol, 25-hydroxyergocalciferol, cholecalciferol, and ergocalciferol. The more preferred Vitamin D compounds useful in the present invention include calcidiol and 25-hydroxyergocalciferol. The most preferred Vitamin D compound in the present invention is calcidiol.

Embodiments of the present invention comprises methods and/or compositions for various routes of administration of a Vitamin D compound and/or mimics thereof in the dosage amounts as recited herein which can be administered to patients with cancer and/or proliferate conditions for treatment which would include but not limited to pharmaceutical compositions of a Vitamin D compound and/or mimics thereof for topical administration with and/or without a transdermal patch.

Other embodiments of the invention include that each and/or any and/or all of the various embodiments of the invention including any and/or all methods, daily dosing, dosage forms, pharmaceutical compositions and the use of Vitamin D compounds and/or mimics thereof, for the manufacture of a medicament for use in treating cancer and/or proliferative conditions in humans as recited herein, may be in any combination and/or non-combination as preferred.

The Institute of Medicine of the National Academy of Sciences teaches the Tolerable Upper Intake Level (UL) for Vitamin D is only 100 mcg per day. The National Cancer Institute at the National Institute of Health teaches that patients with cancer cautiously use or eliminate medications that contain Vitamin D. Various methods, but definitely not administering a Vitamin D compound at very high and/or extremely high daily dose levels in the amounts as recited herein, to patients with proliferative conditions and/or cancers for treatment have been taught with inadequate success to overcome the problem of hypercalcemia and/or unmanageable and/or unsustainable hypercalcemia when administering Vitamin D compounds to patients with proliferative conditions and/or cancers. The methods include escalating the daily dose gradually from an initial small daily dose amount to a higher daily dose amount which said higher dose amount does not even approximate or come close to the dose amounts as recited herein for the present invention and another method previously tried with inadequate results is pulse dosing, that is nondaily dosing which again the pulse dosing amounts does not even approximate or come close to the dose amounts as recited herein for the present invention. There are teachings that Vitamin D can increase blood calcium levels which can lead to hypercalcemia which can be fatal especially in cancer patients and/or in patients with proliferative conditions and this has been a very serious concern for the use of Vitamin D compounds in treatment of patients with cancers and/or proliferative conditions that thereby by not veering too far away from any and/or all of the above said teachings which said teachings are very strong teachings whereby until this invention there has not been a step and/or proposal for treatment for any and/or all cancers and/or proliferative diseases in humans, with very high and/or extremely high novel daily dose amounts as recited herein of a vitamin D compound which again is far beyond and/or extremely far beyond the said daily Tolerable Upper Intake Level dose of Vitamin D of 100 mcg. The surprising and unexpected teachings and the surprising and unexpected results of the new teachings of this invention includes that for patients with cancer and/or proliferative diseases, methods and/or compositions for treatment can be administered to said patients with a Vitamin D compound and/or mimics thereof with a daily dose amount far beyond and/or extremely far beyond the said daily Tolerable Upper Intake Level dose as recited herein or any other daily dose amount that has been attempted or proposed and the methods of very high and/or extremely high dose amount as recited herein for the treatment of patients with cancer and/or proliferative conditions can be accomplished without causing hypercalcemia and/or unmanageable and/or unsustainable hypercalcemia and furthermore said treatment can be successful in one or more aspects and/or totally successful. Further new teachings of this invention includes that patients with cancer and/or proliferative conditions can be administered treatment with very high and/or extremely high daily loading doses amounts as recited herein of a Vitamin D compound and/or mimics thereof and if treatment is not successful and/or adequate before hypercalcemia and/or unmanageable and/or unsustainable hypercalcemia develops in said patient, then once hypercalcemia is resolved in said patient then low daily doses of Vitamin D compound ranging from 1 mcg or less to 0.25 mg or from 1 mcg to 1.25 mg or more of Vitamin D compound and/or mimic thereof can be administered to the patient and then said low daily dose can be gradually escalated to the amounts as recited herein of the very high and/or extremely high loading dose amount until the treatment is successful or until hypercalcemia and/or unmanageable and/or unsustainable hypercalcemia redevelops and then this method can be repeated as necessary. Another surprising and unexpected new teaching of the present invention is that cancer patients and/or patients with proliferative diseases can be treated adequately and/or successfully with very high and/or extremely high daily loading doses in the amounts as recited herein of a Vitamin D compound and/or mimics thereof and that these patients with cancers and/or proliferative conditions with the treatment as described herein will not develop hypercalcemia and/or will not develop hypercalcemia to the extent as in other humans without cancer and/or proliferative conditions who would be administered the same extremely high daily loading dose amounts as recited herein of a Vitamin D compound and/or mimics thereof. All of the above including the said very strong conventional teachings and the new teaching of the invention is equally applicable to each and/or all embodiments of the invention including any and/or all methods, daily dosing, dosage forms, pharmaceutical compositions and the use of Vitamin D compounds and/or mimics thereof, for the manufacture of a medicament for use in treating cancer and/or proliferative conditions in humans as recited herein which may be in any combination and/or non-combination as preferred.

### DETAILED DESCRIPTION OF THE INVENTION

One embodiment of the invention is a method of treatment comprising the administration of a Vitamin D compound and/or mimics thereof in a daily dose amount of 0.25 mg to 500 gm and preferably from 1.25 mg to 500 mg and even more preferably from 2.5 mg to 350 mg and most preferably from 12.5 mg to 50 mg to humans with proliferative conditions and/or cancers.

Another embodiment of the invention is a method of treatment comprising the administration of a vitamin D compound and/or mimics thereof in a daily dose amount of 0.25 mg to 500 gm and preferably from 1.25 mg to 500 mg and even more preferably from 2.5 mg to 350 mg and most preferably from 12.5 mg to 50 mg to humans with proliferative conditions and/or cancers and if hypercalcemia and/or unmanageable and/or unsustainable hypercalcemia develops before the treatment is satisfactorily successful and/or adequate and once hypercalcemia is resolved, then low daily dose amount ranging from lmcg or less to 0.25 mg or from 1 mcg to 1.25 mg or more of Vitamin D compound and/or mimics thereof is administered to said patient for treatment and then said low daily dose is gradually escalated to the loading dose in the amounts as recited herein which then can be administered for treatment to said patient until the treatment is successful and/or adequate or until hypercalcemia and/or unmanageable and/or unsustainable hypercalcemia redevelops and this method can be repeated as necessary.

Another embodiment of invention includes compositions comprising a Vitamin D compounds and/or mimics thereof with an acceptable pharmaceutical carrier in a daily dose amount of 0.25 mg to 500 gm and preferably from 1.25 mg to 500 mg and even more preferably from 2.5 mg to 350 mg and most preferably from 12.5 mg to 50 gm, alone, and/or in combination with other anticancer agents and/or antihypercalcemic agents in amounts that are therapeutically effective for treatment of humans with proliferative diseases and/or cancers.

Another embodiment the invention is a dosage form comprising a Vitamin D compound and/or mimics thereof which may be with an acceptable pharmaceutical carrier administered to humans with proliferative conditions and/or cancer for treatment in a daily dose amount of 0.25 mg to 500 gm and preferably from 1.25 mg to 500 mg and even more preferably from 2.5 mg to 350 mg and most preferably from 12.5 mg to 50 mg.

The preferred Vitamin D compounds useful in the present invention include calcidiol, 25-hydroxyergocalciferol, cholecalciferol, and ergocalciferol. The more preferred Vitamin D compounds useful in the present invention include calcidiol and 25-hydroxyergocalciferol. The most preferred Vitamin D compound in the present invention is calcidiol.

A preferred embodiment of the invention is a method of treatment comprising the administration of a vitamin D compound and/or mimics thereof to humans with proliferative conditions and/or cancers in the most preferred daily dose amount from 12.5 mg to 50 mg and where the preferred vitamin D compound is calcidiol, 25-hydroxyergocalciferol, cholecalciferol, and/or ergocalciferol.

A more preferred embodiment of the invention is a method of treatment comprising the administration of calcidiol in a daily dose amount from 12.5 mg to 50 mg to humans with proliferative conditions and/or cancers.

An even more preferred embodiment of the invention is a pharmaceutical composition comprising a Vitamin D compound and/or mimics thereof in a pharmaceutical dosage form containing from 0.25 mg to 500 gm and preferably from 1.25 mg to 500 mg and even more preferably from 2.5 mg to 350 mg and most preferably from 12.5 mg to 50 mg in combination with a therapeutic effective amount of another anticancer agent with an acceptable pharmaceutical carrier.

Other even more preferred embodiments of the invention are pharmaceutical compositions comprising a Vitamin D compound and/or mimics thereof in the amounts recited herein for topical administration which may be with a pharmaceutical carrier and with and/or without a transdermal patch in the dosage amounts as recited herein. Topical compositions and/or transdermal patches which has the added advantages of providing controlled delivery of a Vitamin D compound to the body in the daily dose amounts as recited herein and may be more effective for patients including but not limited to patients with malabsorption and/or inadequate absorption and/or undetermined adequacy of absorption in the gastrointestinal tract. Following are non-limiting examples comprising topical and/or transdermal compositions with a Vitamin D compound and/or mimics thereof in the daily doses amounts as recited herein. The transdermal patches may be of various forms know in the art such as having an adhesive layer surrounded by a temporary liner and a backing with a reservoir such as there is a drug layer which is a liquid compartment containing a drug solution or suspension separated by the adhesive layer. This patch may also be backed by the backing layer. Here drug reservoir is totally encapsulated in a shallow compartment molded from a drug impermeable metallic plastic laminates, the another side of this so-called rate controlling membrane made up of polymeric membrane like Ethyl Vinyl Acetate. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel. The dosage forms and/or pharmaceutical compositions for the topical and/or transdermal administration of a Vitamin D compound and/or mimics thereof in the amounts as recited herein include powders, sprays, ointments, pastes, creams, lotions, gels, solutions, patches and inhalants. The active Vitamin D compound may be mixed under sterile conditions with a pharmaceutically-acceptable carrier, and with any preservatives, buffers, or propellants which may be required. The ointments, pastes, creams and gels may contain, in addition to Vitamin D compound of the present invention, excipients, such as animal and vegetable fats, oils, waxes, paraffins, starch, tragacanth, cellulose derivatives, polyethylene glycols, silicones, bentonites, silicic acid, talc and zinc oxide, and the like or mixtures thereof.

The most preferred embodiments of the invention are pharmaceutical compositions comprising a Vitamin D compound and/or mimics thereof in the amounts recited herein for topical administration which may be with pharmaceutical carrier with and/or without a transdermal patch in the dosage amounts as recited herein in combination with a therapeutic effective amount of another anticancer agent pursuant and/or in accordance to the above non-limiting examples of topical and/or transdermal compositions.

A large number of Vitamin D compounds are known and can be used in the practice of the invention. Vitamin D compounds of the present invention include, but are not limited to, the analogs, precursors, homologs and derivatives of Vitamin D compounds.

The Institute of Medicine of the National Academy of Sciences teaches the Tolerable Upper Intake Level (UL) for Vitamin D is only 100 mcg per day. The National Cancer Institute at the National Institute of Health teaches that patients with cancer cautiously use or eliminate medications that contain Vitamin D. Various methods, but definitely not administering a Vitamin D compound at very high and/or extremely high daily dose levels in the amounts as recited herein, to patients with proliferative conditions and/or cancers for treatment have been taught with inadequate success to overcome the problem of hypercalcemia and/or unmanageable and/or unsustainable hypercalcemia when administering Vitamin D compounds to patients with proliferative conditions and/or cancers. These methods include escalating the daily dose gradually from an initial small daily dose amount to a higher daily dose amount and said higher dose amount in this method does not even approximate or come close to the dose amounts as recited herein for the present invention and another method previously tried with inadequate results is pulse dosing, that is nondaily dosing which again the pulse dosing amounts does not even approximate or come close to the dose amounts as recited herein for the present invention. There are teachings that Vitamin D can increase blood calcium levels which can lead to hypercalcemia which can be fatal especially in cancer patients and/or in patients with proliferative conditions and this has been a very serious concern for the use of Vitamin D compounds in treatment of patients with cancers and/or proliferative conditions that thereby by not veering too far away from any and/or all of the above said teachings which said teachings are very strong teachings whereby until this invention there has not been a step and/or proposal for treatment for any and/or all cancers and/or proliferative diseases in humans, with very high and/or extremely high novel daily dose amounts as recited herein of a Vitamin D compound and/or mimics thereof which again is far beyond and/or extremely far beyond the said daily Tolerable Upper Intake Level dose of Vitamin D of 100 mcg. The surprising and unexpected teachings and the surprising and unexpected results of the new teachings of this invention includes that for patients with cancer and/or proliferative diseases, methods and/or compositions for treatment can be administered to said patients with a Vitamin D compound and/or mimics thereof with a daily dose amount far beyond and/or extremely far beyond the said Tolerable Upper Intake Level dose amount as referred to herein and/or any other daily dose amount that has been attempted or proposed and the methods of very high and/or extremely high dose amount as recited herein for the treatment of patients with cancer and/or proliferative conditions can be accomplished without causing hypercalcemia and/or unmanageable and/or unsustainable hypercalcemia and furthermore said treatment can be successful in one or more aspects and/or totally successful. Further new teachings of this invention includes that patients with cancer and/or proliferative conditions can be administered treatment with very high and/or extremely high daily loading doses amounts as recited herein of a Vitamin D compound and/or mimics thereof and if treatment is not successful and/or adequate before hypercalcemia and/or unmanageable and/or unsustainable hypercalcemia develops in said patient, then once hypercalcemia is resolved in said patient then low daily doses of Vitamin D compound and/or mimics thereof ranging from lmcg or less to 0.25 mg or from lmcg to 1.25 mg or can be administered to the patient and then said low daily dose can be gradually escalated to the amounts as recited herein of the very high and/or extremely high loading dose until the treatment is successful or until hypercalcemia and/or unmanageable and/or unsustainable hypercalcemia redevelops and then this method can be repeated as necessary. Another surprising and unexpected new teaching of the present invention is that cancer patients and/or patients with proliferative diseases can be treated adequately and/or successfully with very high and/or extremely high daily loading doses in the amounts as recited herein of a Vitamin D compound and/or mimics thereof and that said patients with cancers and/or proliferative conditions treated as described herein will not develop hypercalcemia and/or will not develop hypercalcemia to the extent as in other humans without cancer and/or proliferative conditions who would be administered the same very high and/or extremely high daily loading dose amounts as recited herein of a Vitamin D compound and/or mimics thereof. All of the above including the said very strong conventional teachings and the new teaching of the invention is equally applicable to any and/or all embodiments of the invention including any and/or all methods, daily dosing, dosage forms, pharmaceutical compositions and the use of Vitamin D compounds and/or mimics thereof, for the manufacture of a medicament for use in treating cancer and/or proliferative conditions in humans as recited herein which may be in any combination and/or non-combination as preferred.

### Definitions

The following definitions will help with an understanding of the terms used in this specification.

The terms “Vitamin D compound” and/or “Vitamin D” includes cholecalciferol also known as Vitamin D3 and ergocalciferol, also known as Vitamin D2, 25-hydroxyergocalciferol, also known as 25-hydroxyvitamin D₂, calcidiol, also known as 25-hydroxyvitamin D3 and calcifediol, calcitriol also known as 1alpha,25-dihydroxyvitamin D (1.alpha., 25-dihydroxyvitamin D.sub.3), the 3-hydroxylated dihydrotachysterol.sub.2, the 1.alpha.-hydroxylated alfacalcidol (1.alpha.-hydroxyvitamin D.sub.3) and the terms also includes any substance that has an affinity for the Vitamin D Receptor and/or Vitamin D-Binding Protein; the term also includes any of the family of secosteroids with antirhichitic activity and/or antineoplastic activity, which would again include ergocalciferol and cholecalciferol , any of their precursor molecules such as ergosterol (7-dehydro-22-dehydro-24-methyl-cholesterol) and 7 dehydrocholesterol, and as used herein the terms includes any compound which activates the Vitamin D Receptor, by binding or otherwise, either in its form of administration or in a form to which it is converted by processing by the human body. The terms includes each of Vitamins D.sub.1, D.sub.2, D.sub.3, D.sub.4 and D.sub.5 and the various known analogues, precursors, homologs and derivatives thereof and any other agent that has Vitamin D activity and/or is an agonist thereof and/or that thereby increases the rate of apoptosis in cancer cells . The terms further includes as well as the numerous natural and synthetic Vitamin D analogs, precursors, homologs and derivatives, of any Vitamin D compound described herein and further non-limiting examples are set forth in Bouillon et. al, Endocrine Reviews 16: 200-257,1995. Vitamin D compounds of the present invention include, but are not limited to, the analogs, precursors, homologs and derivatives of Vitamin D compounds of any and/or all of the Vitamin D compounds and/or mimics thereof that are described herein and it is contemplated that not only presently available Vitamin D analogues, precursors, homologs and derivatives and/or mimics thereof but also Vitamin D analogues, precursors, homologs and derivatives and/or mimics thereof introduced in the future will be useful according to the present invention.

The term “mimic” as used herein is intended to refer to non-secosteroidal Vitamin D mimic compounds. In general, these non-secosteroidal Vitamin D mimics are compounds that do not structurally fall within the class of compounds generally known as Vitamin D compounds but which modulate the activity of Vitamin D Nuclear Receptors and/or have affinity for the Vitamin D Nuclear Receptors and/or the Vitamin D Binding Protein. Non-limiting examples of such Vitamin D mimics include bis-aryl derivatives disclosed by U.S. Pat. No. 6,218,430 and WO publication 2005/037755. Additional examples of non-secosteroidal Vitamin D mimic compounds suitable for the present invention can be found in U.S. Pat. Nos. 6,831,106; 6,706,725; 6,689,922; 6,548,715; 6,288,249; 6,184,422, 6,017,907, 6,858,595 and 6,358,939.

The term “other anticancer agents” include all known other anticancer agents and/or any agent which potentially may be anticancer agent and/or anticancer agent which may be developed in the future including but not limited to the following non-limiting examples of anticancer agents: Abiraterone Acetate Abitrexate (Methotrexate) Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine,Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Amboclorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, BendamustineHydrochloride, BEP, Bevac izumab, Bexarotene, Bexxar (Tositumomab and I 131 Iodine Tositumomab), Bleomycin, Bortezomib, Bosulif (Bosutinib), Bosutinib, Brentuximab Vedotin, Cabazitaxel, Cabozantinib-S-Malate, CAF, Campath (Alemtuzumab), Camptosar (Irinotecan Hydrochloride), Capecitabine, CAPDX, Carboplatin, CARBOPLATIN-TAXOL, Carfilzomib, CeeNU (Lomustine), Cerubidine (Daunorubicin Hydrochloride), Cervarix (Recombinant HPV Bivalent Vaccine), Cetuximab, Chlorambucil, CHLORAMBUCIL-PREDNISONE, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen(Dactinomycin), Crizotinib, CVP, Cyclophosphamide, Cyfos (Ifosfamide), Cytarabine, Cytarabine, Liposomal, Cytosar-U (Cytarabine), Cytoxan (Cyclophosphamide), Dabrafenib, Dacarbazine, Dacogen (Decitabine), Dactinomycin, Dasatinib, Daunorubicin, Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine, Recombinant, HPV Quadrivalent Vaccine, Recombinant, Hycamtin (Topotecan Hydrochloride), Ibritumomab Tiuxetan, Ibrutinib, ICE, Iclusig (Ponatinib Hydrochloride), Ifex (Ifosfamide), Ifosfamide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc),Sipuleucel-T, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), TAC, Tafinlar (Dabrafenib), Talc, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and I 131 Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VeIP, Velban (Vinblastine Sulfate), Velcade (Bortezomib), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), XELOX, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza stat), Zometa (Zoledronic Acid), Zytiga (Abiraterone Acetate).

The term “cancer,” as used herein, is intended to refer to any known cancer, and includes, the following non-limiting examples : Acute Lymphoblastic Leukemia, Adult; Acute Lymphoblastic Leukemia, Childhood: Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stein Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood; Carcinoid Tumor, Childhood; Carcinoid Tumor,Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral AstrocytomalMalignant Glioma, Childhood; Cervical Cancer; Childhood Cancers: Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stern; Glioma, Childhood Visual pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; liodgkin's Lymphoma During Pregnancy; Hypopharyngeal Cancer; Hypothalamic and Visual pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood: Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver, Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastotria, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Ca Neoplasm/Multiple Myeloma; Pleuropulmonary Blastoma: Pregnancy and Breast Cancer: Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcorna, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma,; Childhood; Sarcoma, Soft Tissue, Adult; Sarcoma, Soft Tissue, Childhood; Sezary Syndrome; Skin Cancer; Skin Cancer, Childhood: Skin Cancer (Melanoma); Skin Carcinoma, Merkel Cell; Small Ca Lung Cancer; Small intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Th.ymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual pathway and Hypothalamic Glioma, Childhood; Vulvar Cancer; Waldenstrom's Macroglobulinemia; and Wilms' Tumor. See Fishman et al., 1985, Medicine, 2d Ed., J. B. Lippincott Co., Philadelphia, Pa. and Murphy et al., 1997, Informed Decisions: The Complete Book of Cancer Diagnosis, Treatment, and Recovery, Viking Penguin, New York, N.Y., for a review of such disorders and non-limiting examples of cancer. All stages and/or forms of any and/or all of the above cancers are included regardless of the different stage classifications known and/or the different form classifications known and furthermore precancerous conditions of any an lor all of the above cancers are also included and/or cancers that the treatments is improved by this invention in comparison to other available treatments are included and/or furthermore especially any and/or all of the above cancers that are resistant and/or refractory and/or do not respond satisfactory to other known availably treatments and/or cancers for which there are no available effective treatments for such as terminal cancers are also included, Furthermore once a patient is diagnosed as being cancer free after having cancer, all methods, daily dosing, dosage forms, pharmaceutical compositions and the use of Vitamin D compounds, for the manufacture of a medicament for use in treatment as described herein, that is any and/or all of the embodiments of the invention , also can be used in the preventative treatment so that the cancer does not reoccur and/or is delayed and/or ameliorated in any aspect if in the unlikely event that the cancer does reoccur.

The term or terms “proliferative condition” and/or “conditions” and/or “proliferative disease” and/or “proliferative diseases” includes all of the various conventional and/or common understandings of the terms which would include any known proliferative condition and/or proliferative disease which includes cancer and said terms would further include as non-limiting examples, benign hyperplasia of the prostate and benign hyperplasia of the breast including usual hyperplasia and atypical hyperplasia of the breast and psoriasis and/or in addition also said terms may include adenomatous polyps of the colon and rectum even though adenomatous polyps of the colon and rectum may not necessarily be conventional considered proliferative conditions. Said terms further include especially any and/or all of the above proliferative conditions that are resistant and/or refractory and/or do not respond satisfactory to other known availably treatments and/or proliferative conditions for which there are no available effective treatments and /or proliferative conditions that the treatments is improved by this invention in comparison to other available treatments are included.

The term “antihypercalcemic agent” includes any known antihypercalcemic agents and/or any agent which potentially may be antihypercalcemic agent and/or any antihypercalcemic agent which may be developed in the future and further includes as non-limiting examples, glucocorticoids, gallium nitrate, plicamycin, bisphosphonates etidronate disodium and pamidronate disodium, calcitonin, furosemide and zoledronic acid.

The terms “patients” and/or “patient” refers to only humans who may have cancer and/or a proliferative condition.

The terms “treating” and/or “treatment” includes all the various conventional and/or common understandings of the terms and non-limiting examples include slowing the progress and/or rate of progress of the disease and/or condition and/or any and/or all symptoms thereof and/or amelioration of any and/or all symptoms thereof and/or full and/or partial cure of the condition and/or disease and/or the prevention and/or slowing the reoccurrence of the disease and/or condition.

The terms “loading dose” and/or the “amounts as recited herein” and/or “loading dose” and/or “loading dose amounts” and/or “daily dose amount” refers to a daily dose amount of 0.25 mg to 500 gm and preferably from 1.25 mg to 500 mg and even more preferably from 2.5 mg to 350 mg and most preferably from 12.5 mg to 50 mg unless otherwise it is clear from the context and/or necessary to within any particular embodiment of the present invention. An example of an exception to the daily dose amount is from an embodiment of the invention where once hypercalcemia is resolved, then low daily dose amount ranging from 1 mcg or less to 0.25 mg or from 1 mcg to 1.25 mg or more of Vitamin D compound and/or mimics thereof is administered to said patient for treatment and then said low daily dose is gradually escalated to the loading dose in the amounts as recited herein which then can be administered for treatment to said patient until the treatment is successful and/or adequate or until hypercalcemia redevelops and this method can be repeated as necessary whereby in this instance the “low ‘daily dose amount’” refers to 1 mcg or less to 0.25 mg or from 1 mcg to 1.25 mg or more and again in this particular instance not to the loading dose amount.

The singular terms “a,” “an,” and “the” include plural references unless context clearly indicates otherwise.

Any singular term used herein may also be considered as plural term and any plural term used herein may also be considered a singular term unless otherwise it is clear from the context and/or necessary to within any particular embodiments of the present invention and non-limiting examples are that patients with cancers may have one or more cancers and/or patients with cancer may have just one cancer or more than one cancer and another non-limiting example is that a compound may be a single compound or may be a number of compounds more than one.

It also is specifically understood that any numerical value and/or numerical amounts and/or range of a numerical values recited herein includes all values from the lower value to the upper value, i.e., all possible combinations and/or non-combinations of numerical values between and including the lowest value and/or amount and the highest value and/or amount enumerated are to be considered to be expressly stated in this application. For example, if a concentration range and/or a beneficial effect range and/or amounts is stated as 1% to 50%, it is intended that values such as 2% to 40%, 10% to 30%, or 1% to 3%, etc., are expressly enumerated in this specification. These are non-limiting examples of what is specifically intended.

It is specifically understood that e ems of the invention include that any and/or all of the various embodiments of the invention including any and/or all methods, daily dosing, dosage forms, pharmaceutical compositions and the use of Vitamin D compounds and/or mimics thereof, for the manufacture of a medicament for use in treating cancer and/or proliferative conditions in humans as recited herein may be in any combination and/or non-combination as preferred and non-limiting examples include that the dosage amounts of a Vitamin D compound and/or mimics thereof in aerosol compositions and/or transdermal patches and/or topical compositions may be the loading dose amounts as recited herein and then if hypercalcemia and/or unmanageable and/or unsustainable hypercalcemia develops in a patient with cancer and/or proliferative conditions and once the hypercalcemia is resolved in said patient then the amounts of the low daily doses from 1 mcg or less to 0.25 mg or from 1 mcg to 1.25 mg or more of Vitamin D compound and/or mimics thereof can be the amounts in other aerosol compositions and/or transdermal patches and/or topical compositions which can be administered to said patient and then said low daily dose amounts can be gradually escalated to the loading dose amounts as recited herein in other aerosol compositions and/or transdermal patches and/or topical compositions which can be administered to said patient for treatment. Further non-limiting examples of embodiments of the invention in regard to any combination and/or non-combination is a method of treatment comprising the administration of a Vitamin D compound and/or mimics there to humans with proliferative conditions and/or cancers in the most preferred daily dose amounts as recited herein and where there may be an exact daily dose amount of 12.5 mg and/or where there may be an exact daily dose amount of 30 mg and/or where there may be an exact daily dose amount of 50 mg and/or where there may be an exact daily dose amount anywhere else within the specified range and/or referred to amounts and/or where the preferred Vitamin D compound is calcidiol, cholecalciferol 25-hydroxyergocalciferol, and/or ergocalciferol and/or where the most preferred Vitamin D compound is calcidiol. It is specifically understood any combination and/or non-combination which may be possible within the herein described invention may be selected as desired and/or preferred, consistent with the herein specifications in regard to additional embodiments of the invention.

It is specifically understood that all examples recited herein are non-limiting examples cited for the purposes of illustration.

Embodiments of the present invention include methods comprising various routes of administration for treatment to a patient with cancer and/or proliferate condition of a Vitamin D compound and/or mimics thereof in the dosage amounts as recited herein and examples of routes of administration which can be used include injection subcutaneous, intravenous, parenterally, intraperitoneally, further examples of routes of administration include oral, inhalation, rectal, transdermal or via bladder instillation. The pharmaceutical preparations are, of course, given by forms suitable for each administration route. For example, these preparations are administered in tablets or capsule form, by injection, infusion, inhalation, lotion, ointment, suppository, etc. Oral and/or topical and/or transdermal administration is preferred. The injection can be bolus or can be continuous infusion. Depending on the route of administration, the Vitamin D compound and/or mimics thereof can be coated with or disposed in a selected material to protect it from natural conditions which may detrimentally effect its ability to perform its intended function. The Vitamin D compound and/or mimics thereof can be administered alone, or in conjunction with either another agent useful in the treatment.

Examples of the dosage forms and/or compositions of the invention may be an oral, intravenous, intramuscular, topical, subcutaneous, transdermal, sublingual, intranasal, or other preparation, but in particular disclosed embodiments the pharmaceutical dosage form and/or composition is preferably an oral dosage form and/or composition, such as a capsule or tablet and/or other particular disclosed embodiments the pharmaceutical dosage form and/or composition is preferably a topical and/or transdermal composition.

Examples of compositions of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount as recited herein of a Vitamin D compound and/or mimics thereof as an active ingredient. A compound may also be administered as a bolus, electuary or paste.

The daily dose in the amounts as recited herein of the Vitamin D compound and/or mimics thereof may be in various dosage forms including liquid dosage forms and/or capsule forms for oral administration of the Vitamin D compound which may include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Any daily dose amount for any embodiment of the invention may be divided as preferred into equal or unequal daily dose amounts and an example of which is in the instant where the daily dose amount is 50 mg said amount may be divided into 5 equal dose amounts of 10 mg each which may be administered as preferred in any time interval as preferred during the day for a total of 50 mg which equals the daily dose amount of 50 mg in this instance.

In addition to inert diluents, the oral compositions can include adjuvants such as wetting agents, emulsifying and suspending agents, sweetening, flavoring, coloring, perfuming and preservative agents.

Suspensions, in addition to the active Vitamin D and/or mimic thereof compound may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

In solid dosage forms of the invention for oral administration (capsules, tablets, pills, dragees, powders, granules and the like), the active ingredient may be mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, acetyl alcohol and glycerol monostearate; absorbents, such as kaolin and bentonite clay; lubricants, such a talc, calcium stearate, magnesium stearate, solid polyethylene glycols, sodium lauryl sulfate; buffering agents; coloring agents and mixtures thereof. Solid compositions of a similar type may also be employed as fillers in soft and hard-filled gelatin capsules using such excipients as lactose or milk sugars, as well as high molecular weight polyethylene glycols and the like.

Other embodiments of the invention are pharmaceutical compositions for rectal or administration which may comprise a suppository, which may be prepared by mixing a Vitamin D compound and/or mimic thereof in the amounts as recited herein with one or more suitable nonirritating excipients or carriers comprising, for example, cocoa butter, polyethylene glycol, a suppository wax or a salicylate, and which is solid at room temperature, but liquid at body temperature and, therefore, will melt in the rectum and release the active agent. This embodiment of the invention may have the added advantages of providing controlled delivery of a Vitamin D compound to the body in the daily dose amounts as recited herein and may be more effective for patients including but not limited to patients with malabsorption and/or inadequate absorption and/or undetermined adequacy of absorption in the gastrointestinal tract

Powders and sprays can contain, in addition to a Vitamin D compound and/or mimic thereof, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Other embodiments of the invention are methods of treatment comprising a Vitamin D compound and/or mimics thereof in the amounts as recited herein, which is to be administered alternatively by aerosol to humans with cancer and/or proliferate conditions which especially though not the only method of administration may be more effective for cancers of the respiratory tract,

Other embodiments of the invention are pharmaceutical aerosol composition comprising a Vitamin D compound and/or mimics thereof in the amounts as recited herein, in combination with any and/or all of the following including an aqueous aerosol, liposomal preparation or solid particles containing the compound and a nonaqueous (e.g., fluorocarbon propellant) suspension could be used and sonic nebulizers are preferred because they minimize exposing the agent to shear, which can result in degradation of the compound and ordinarily an aqueous aerosol is made by formulating an aqueous solution or suspension of the agent together with conventional pharmaceutically-acceptable carriers and stabilizers and the carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions. The above are non- limiting examples. The embodiments of the invention with aerosol may have the added advantages of providing controlled delivery of a Vitamin D compound to the body in the daily dose amounts as recited herein and may be more effective for patients including but not limited to patients with malabsorption and/or inadequate absorption and/or undetermined adequacy of absorption in the gastrointestinal tract

Suspensions, in addition to the active Vitamin D compound and/or mimic thereof may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Other embodiments of the invention include pharmaceutical compositions suitable for parenteral administration which comprises a Vitamin D compound and/or mimics thereof in the amounts recited herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient and/or suspending and/or thickening agents and the like.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Injectable depot forms are made by forming microencapsule matrices of a Vitamin D compound and/or mimic thereof in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

Pharmaceutically acceptable carriers useful in this disclosure of the recited compositions may include inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof and these compositions may also contain adjuvants such as preservatives, wetting agents, emulsifying agents and dispersing agents and antioxidants including vitamin E and prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminium monostearate and gelatin the pharmaceutically acceptable carriers useful in this disclosure are conventional. Remington's Pharmaceutical Sciences, by E. W. Martin, Mack Publishing Co., Easton, Pa., 15th Edition (1975), describes non-limiting examples of pharmaceutical carriers and/or compositions and/or formulations suitable for pharmaceutical delivery and/or suitable as carriers of the compositions of the herein disclosed invention.

Various pharmaceutical carriers and/or compositions and/or formulations suitable for pharmaceutical delivery and/or suitable as carriers for the compositions of the herein disclosed invention are well known in the art.

Those skilled in the art will recognize, many equivalents of the specific embodiments of the invention described herein. Such equivalents are specifically intended to be encompassed in the herein specifications. The herein recited description has outlined, in specifics and/or in general, the featured aspects of the invention and is to serve as an aid to better understanding the more complete detailed description in reference to such, there is to be a clear understanding that the present invention is not limited to the method and/or detail of manufacture and/or use of a compound and/or chemical composition and/or use of a compound, for the manufacture of a medicament for use in treatment and/or application and/or use described herein wherefore any other variation of manufacture and/or use of a compound and/or chemical composition and/or use of a compound, for the manufacture of a medicament for use in treatment and/or method, and/or use and/or application should be considered apparent as an alternative embodiments of the present invention. Other advantages and a fuller appreciation of the specific adaptations, compositional variations and chemical and physical attributes of this invention will be gained upon examination of the detailed description. Also, it is understood that the phraseology and terminology used herein are for the purpose of description and should not be regarded as limiting wherefore the use of “including,” “having,” and “comprising,” and variations thereof here in is meant to encompass the items listed thereafter and equivalents thereof.

All patents, publications and references cited herein are hereby fully incorporated by reference. In case of conflict between the present disclosure and incorporated patents, pUblications and references, the present disclosure should control.

1. Having described the invention I claim a method of treatment comprising administering a vitamin d compound and/or mimics thereof in a daily dose amount of 0.25 mg to 500 gm and preferably from 1.25 mg to 500 mg and even more preferably from 2.5 mg to 350 mg and most preferably from 12.5 mg to 50 mg to humans with proliferative conditions and/or cancers.

2. The method of claim 1 where the Vitamin D compound is selected from: calcidiol, 25-hydroxyergocalciferol, cholecalciferol, and ergocalciferol.

3. The method of claim 1 where the Vitamin D compound is calcidiol.

4. The method of claim 1 where the cancers is selected from the following cancers: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stern Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual pathway and Hypothalamic Glioma, Childhood; Brain Tumor, Childhood (Other); Breast Cancer; Breast Cancer and Pregnancy: Breast Cancer, Childhood; Breast Cancer, Male; Bronchial Adenomas/Carcinoids, Childhood; Carcinoid Tumor, Childhood; Carcinoid Tumor, Gastrointestinai: Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma, Childhood Brain Stem; Glioma, Childhood Visual pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual pathway Glioma, Childhood; Intraocular Melanoma: Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver, Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's, Childhood; Lymphoma, Hodgkin's During Pregnancy; Lymphoma, Non-Hodgkin's, Adult; Lymphoma, Non-Hodgkin's, Childhood; Dymphoma, Non-Hodgkin's During Pregnancy; Lymphoma, Primary Central Nervous System; Macroglobulinemia, Waldenström's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood; Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple. Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual pathway and Hypothalamic Glioma, Childhood; Vulvar Cancer; Waldenström's Macroglobulinemia; and Wilms' Tumor.

5. The method of claim 1 where the dosage amount is from 12.5 mg to 50 mg.

6. The method of claim 1 where the cancer is treatment resistant.

7. The method of claim 1 where the cancer is terminal.

8. A composition comprising a Vitamin D compounds and/or mimics thereof with an acceptable pharmaceutical carrier in a daily dose amount of 0.25 mg to 500 gm and preferably from 1.25 mg to 500 mg and even more preferably from 2.5 mg to 350 mg and most preferably from 12.5 mg to 50 gm, for treatment of humans with proliferative diseases and/or cancers.

9. The composition of claim 8 where the Vitamin D compound and/or mimics thereof is selected from: calcidiol, 25-hydroxyergocalciferol, cholecalciferol, and ergocalciferol.

10. The composition of claim 8 where the Vitamin D compound and/or mimics is calcidiol.

11. The composition of claim 8 where the Vitamin D compound and/or mimics thereof is in a daily dose amount from 12.5 mg to 50 mg.

12. The composition of claim 8 where the pharmaceutical carrier is a transdermal patch.

13. The composition of claim 8 where the pharmaceutical carrier is a capsule.

14. The composition of claim 8 where the pharmaceutical carrier is a tablet.

15. The composition of claim 8 where the pharmaceutical carrier is an aerosol.

16. The composition of claim 8 where the pharmaceutical carrier is a liquid.
